(19) United States
(12) Patent Application Publication
(10) Pub. No.: US 2010/0121325 A1
(43) Pub. Date: May 13, 2010

(54) Hybrid intramedullary fixation assembly and method of use

(76) Inventors: Jeff Tyber, Bethlehem, PA (US); Jamy Gannoe, West Milford, NJ (US); Chris Digiovanni, Barrington, RI (US)

Correspondence Address: WARD & OLIVO, SUITE 300, 382 SPRINGFIELD AVENUE, SUMMIT, NJ, 07901, US

(21) Appl. No.: 12/587,445

(22) Filed: Oct. 7, 2009

Related U.S. Application Data
(63) Continuation-in-part of application No. 12/456,808, filed on Jun. 23, 2009.
(60) Provisional application No. 61/132,932, filed on Jun. 24, 2008.

(51) Int. Cl. A61B 17/56; A61B 17/80

(52) U.S. Cl. 606/62; 606/286

(57) ABSTRACT

A hybrid intramedullary fixation assembly for joint stabilization is provided and includes a plate member having a plurality of apertures. The plate member includes a first elongated portion and a second curved portion. The assembly includes a plurality of metatarsal screws for coupling the plate member to the first elongated portion and to the metatarsal bone, an intramedullary screw member coupled to the first elongated portion applies compression to the tarsometarsal joint, and a plurality of medial screws coupled to the second curved portion and to the bones in the mid-foot region stabilizes the joint.

### CROSS-REFERENCE TO RELATED APPLICATIONS

This application is a continuation-in-part application of Non-Provisional application Ser. No. 12/456,808, filed Jun. 23, 2009, which claims the benefit of Provisional Application No. 61/132,932, filed Jun. 24, 2008, the entire contents of which are herein incorporated by reference.

### FIELD OF THE INVENTION

This invention relates to the field of orthopedic implant devices, and more particularly, to a hybrid intramedullary fixation assembly comprising a screw and plate fixation assembly used for internal fixation of angled joints, bones and joint reinforcement, such as the bones in the foot.

### BACKGROUND OF THE INVENTION

Orthopedic implant devices, such as intramedullary nails, plates, rods and screws are often used to repair or reconstruct bones and joints affected by trauma, degeneration, injury, deformity and disease, such as injuries to the tarsometatarsal joint caused by accidents or falls or Charcot arthropathy caused by diabetes in some patients.

Injuries to the tarsometarsal joint occur in athletics, from minor twisting injuries when stepping unevenly, to more violent injuries that may occur in motor vehicle accidents or falls, while charcot arthropathy (or Charcot foot) is a destructive process affecting many regions including joints of the foot and ankle in diabetics. This condition causes bony fragmentation, dislocation, and fractures that eventually progresses to foot deformity, bony prominences, ulceration and instability of the foot. Charcot arthropathy can affect any joint in the body but is often seen in the feet affecting the metatarsal, tarsometatarsal and tarsal joints and frequently causes the foot to lose its arch or curvature, and joint stability, thus resulting in “flat footedness” in the mid-foot region.

Surgery is required for the majority of the tarsometarsal injuries. The treatment of tarsometatarsal injuries is usually done by reduction of the fraction or dislocation by means of screws that are inserted internally into the bones across the joints. These can be inserted through multiple punctures made on the skin without resorting to incisions on the foot. On the other hand, early treatment for Charcot foot includes the use of therapeutic footwear, immobilization of the foot and/or non-weight bearing treatment. Surgical treatments include orthopedic fixation devices that fixate the bones in order to fuse them into a stable mass. These orthopedic implant devices realign bone segments and hold them together in compression until healing occurs, resulting in a stable mass.

Various implants have been utilized for surgical treatment, including bone screws. While these devices allow fixation and promote fusion, they do not reinforce the joint nor do they restore the arch in a Charcot foot. Instead, the physician must estimate the arch and manually align the bones and deliver the screws to hold the bones in place, while reducing bone purchase. Intramedullary nails and/or a plate with a lag screw too have deficiencies. These intramedullary nails also do not reconstruct an arch that is lost due to Charcot foot disease nor do they reinforce the tarsometatarsal joint.

Moreover, infections and wound complications are a major concern in aforementioned procedures. Wound closure is technically demanding for the surgeon, and devices that add surface prominence, such as plates or exposed screws, add to the difficulty by requiring greater tissue tension during incision reapproximation. This increases the risk of postoperative wound infections and dehiscence that may ultimately result in limb amputation.

There is therefore a need for a hybrid intramedullary fixation assembly and method of use that overcomes some or all of the previously delineated drawbacks of prior fixation assemblies.

### SUMMARY OF THE INVENTION

An object of the invention is to overcome the drawbacks of previous inventions.

Another object of the invention is to provide a novel and useful intramedullary fixation assembly that may be utilized to treat bones in a mid-foot region.

Another object of the invention is to provide joint reinforcement of the mid-foot region by utilizing a hybrid intramedullary screw and plate assembly.

Another object of the invention is to provide a system for treating deteriorating bones in a mid-foot region.

Another object of the invention is to provide a method for reinforcing the bones in the foot by delivering a plate and screw fixator that can be coupled to bones in a patient's foot.

In a first non-limiting aspect of the invention, an intramedullary fixation assembly for joint stabilization is provided and includes a plate member having a plurality of apertures, where the plate member comprises a first elongated portion and a second curved portion. The assembly further includes a plurality of metatarsal screws for coupling the plate member to the first elongated portion and to the metatarsal bone. An intramedullary screw member coupled to the first elongated portion applies compression to the tarsometarsal joint and a plurality of medial screws coupled to the second curved portion stabilizes the joint.

In a second non-limiting aspect of the invention, a method for reinforcing a tarsometarsal joint in a mid-foot region comprises six steps. Step one includes making a Medial Lis Franc incision in the mid-foot region of the human foot in order to gain access to the tarsometarsal joint. Step two includes Gunstocking the foot to expose the articular surface and removing the articulating cartilage. Step three includes inserting metatarsal screws into the tarsometatarsal plate member and into the metatarsal to anchor the metatarsal screws. Step four includes inserting the intramedullary screws into the tarsometarsal joint and applying compression. Step five includes inserting medial-lateral screws into the bones in the mid-foot region. The sixth step includes closing the incision, thereby reinforcing the tarsometarsal joint in the mid-foot region.

### BRIEF DESCRIPTION OF THE DRAWINGS

A further understanding of the invention can be obtained by reference to a preferred embodiment set forth in the illustrations of the accompanying drawings. Although the illustrated embodiment is merely exemplary of systems and methods for carrying out the invention, both the organization and method of operation of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the invention, reference is now made to the following drawings in which:

FIG. 1 is a perspective view of a fixation system according to a preferred embodiment of the invention.

FIG. 2 is a perspective view of a proximal screw member used in the fixation system shown in FIG. 1 according to the preferred embodiment of the invention.

FIG. 3A is a perspective view of a distal member used in the fixation system shown in FIG. 1 according to the preferred embodiment of the invention.

FIG. 3B is a perspective cross-sectional view of the distal member shown in FIG. 3A according to the preferred embodiment of the invention.

FIG. 4 is a perspective view of the instrument member used in the fixation system shown in FIG. 1 according to the preferred embodiment of the invention.

FIG. 5 is a perspective view of the assembled intramedullary fixation assembly inserted into the bones of a patient's foot according to the preferred embodiment of the invention.

FIG. 6 is a side view of the assembled intramedullary fixation assembly shown in FIG. 5 according to the preferred embodiment of the invention.

FIG. 7 is a flow chart illustrating the method of coupling the intramedullary fixation assembly shown in FIGS. 1-6 to tarsal and metatarsal bones in a patient's foot according to the preferred embodiment of the invention.

FIG. 8 is a perspective view of an assembled intramedullary fixation assembly according to an embodiment of the invention.

FIG. 9 is a perspective top view of an assembled intramedullary fixation assembly shown in FIG. 8 according to an embodiment of the invention.

FIG. 10 is another view of the assembled intramedullary fixation assembly shown in FIGS. 8-9 according to an embodiment of the invention.

FIG. 11 is a perspective front view of a tarsometatarsal plate member used in the fixation assembly shown in FIGS. 8-10 according to the embodiment of the invention.

FIG. 12 is top view of the tarsal-metatarsal plate member shown in FIGS. 8-11 according to an embodiment of the invention.

FIG. 13 is a flow chart illustrating the method of coupling the intramedullary fixation assembly shown in FIGS. 8-12 to bones in the mid-foot region according to an embodiment of the invention.

### DETAILED DESCRIPTION

The invention may be understood more readily by reference to the following detailed description of preferred embodiment of the invention. However, techniques, systems, and operating structures in accordance with the invention may be embodied in a wide variety of forms and modes, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

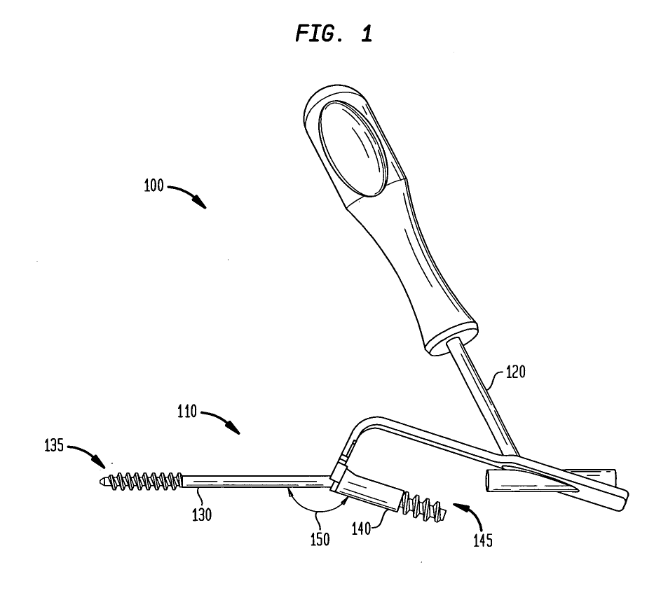

Referring now to FIG. 1, there is shown a fixation system 100 which is made in accordance with the teachings of the preferred embodiment of the invention. As shown, the fixation system 100 includes an intramedullary fixation assembly 110, comprising a proximal screw member 130 and a distal member 140. Proximal screw member 130 is provided on proximal end 135 of assembly 110 and is coupled to a distal member 140 that is provided on the distal end 145 of the fixation assembly 110. Also, proximal screw member 130 makes a fixed angle 150 with distal member 140 and this angle 150 determines the angle for arch restoration. Moreover, fixation system 100 includes instrument 120 that is utilized to couple intramedullary fixation assembly 110 to the bones, in one non-limiting example, in the mid-foot region (not shown). It should be appreciated that in one non-limiting embodiment, intramedullary fixation assembly 110 may be made from a Titanium material, although, in other non-limiting embodiments, intramedullary fixation assembly 110 may be made from SST, PEEK, NiTi, Cobalt chrome or other similar types of materials. It should also be appreciated that intramedullary fixation assembly 110 may be utilized for the internal fixation of other bones in the human body.

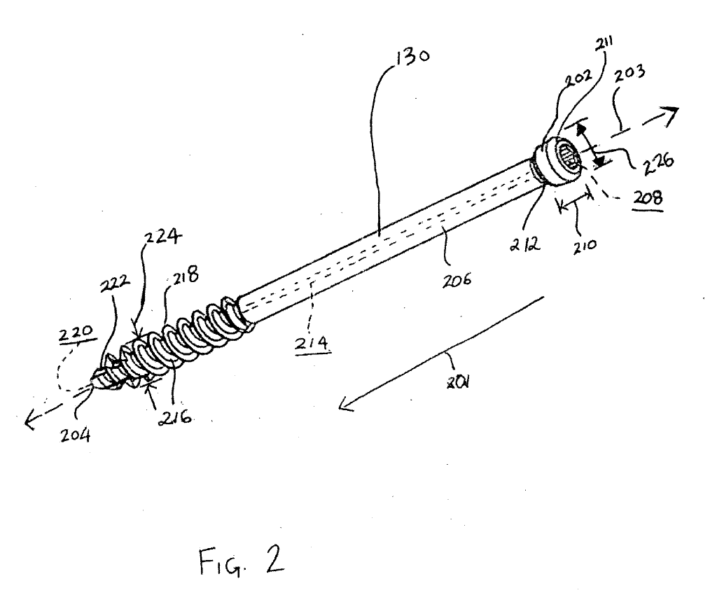

As shown in FIG. 2, proximal screw member 130 is generally cylindrical in shape and extends from first bulbous portion 202 to second tapered end 204. End 204 has a diameter that is slightly smaller than diameter 226 of bulbous portion 202. Additionally, bulbous portion 202 has a taper, such as a Morse taper, with a width that decreases from end 211 to end 212. The taper allows for a locked interference fit with tapered aperture 316 when tapered bulbous portion 202 is combined with tapered aperture 316, shown and described below. Moreover, bulbous portion 202 is generally circular and has a generally hexagonal torque transmitting aperture 208 that traverses length 210 of bulbous portion 202. However, a star-shaped aperture, a square-shaped aperture, or any other shaped aperture may be utilized without departing from the scope of the invention. Torque transmitting aperture 208 is utilized to transmit a torque from bulbous portion 202 to tapered end 204 by rotating bulbous portion 202.

Further, proximal screw member 130 has a first smooth exterior portion 206 extending from end 212 of bulbous portion 202. Portion 206 comprises an internal aperture 214 that longitudinally traverses portion 206 in direction 201. Portion 206 terminates into a second generally tubular portion 216. Portion 216 may comprise internal circular aperture 220 that longitudinally traverses inside portion 216. Internal circular aperture 220 is aligned with apertures 214 and 208 along axis 203 to form a continuous opening (i.e., a cannula) from bulbous portion 202 to end 204. The continuous opening or cannula is provided to interact with a guide wire (not shown) by receiving the guide wire within the continuous opening thereby positioning and locating the proximal member 130. In other non-limiting embodiments, the proximal member 130 may be provided without apertures 220 and 214 (i.e., the proximal member is solid).

Furthermore, tubular portion 216 has a plurality of circular threads, such as threads 218, which are circumferentially disposed on the external surface of portion 216 and, with threads 218 having an external diameter 224. Portion 216 may also be provided with a self-tapping leading edge 222 to provide portion 216 with the ability to remove bone material during insertion of proximal screw member 130 into bone. It should be appreciated that the length of the proximal member 130 may be selected of varying lengths to allow a surgeon to fuse different joints in a foot (not shown).

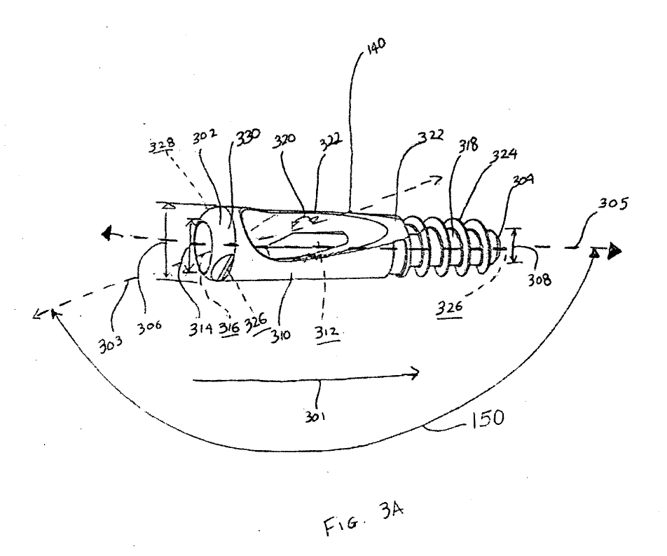

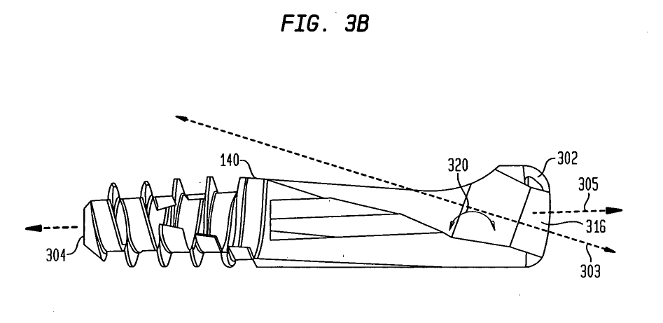

As shown in FIGS. 3A-3B, distal member 140 of the preferred embodiment is generally tubular in shape and tapers from a first end 302 to a second end 304 (i.e. end 302 has a diameter 306 that is slightly larger than diameter 308 of end 304). However, in another non-limiting embodiment, distal member 140 has a constant width from first end 302 to second end 304. Further, first end 302 is generally semi-spherical in shape and has an internal circular aperture 316, which traverses end 302 along direction 301 (i.e. end 302 is generally “donut” shaped). Additionally, circular aperture 316 emanates from surface 322, such that portion 310 has a generally tapered aperture 316 provided in portion 310. Circular aperture 316 comprises slope 320 from first end 302 to end 322 of portion 310. Further, aperture 316 is aligned along axis 303, which is offset from horizontal axis 305 of distal member 140. Axis 303 forms an angle 150 with horizontal axis 305 that determines the angle for arch restoration, as shown in FIG. 3A. Angle 150 may be any angle greater than 90 degrees and less than 180 degrees. Tapered aperture 316 when combined with tapered bulbous portion 202, shown in FIG. 2, creates a locked interference fit between proximal member 130 and distal member 140. First end 302 has a plurality of substantially similar grooves 326 and 328, which form an “L-shape” with surface 330 of end 302. Grooves 326 and 328 are provided to receive instrument 120 of fixation system 100, which is later described. In other non-limiting embodiments, other similar instruments may be provided to be received within grooves 326 and 328.

Distal member 140 further comprises a generally smooth portion 310 coupled to end 302. Portion 310 has a generally hexagonal shaped aperture 312, which opens into aperture 316 and which longitudinally traverses through portion 310 in direction 301. In other non-limiting embodiments, a star-shaped aperture, a square-shaped aperture, or any other shaped aperture may be utilized. Circular aperture 316 has a diameter 314 that is slightly larger than external diameter 224 of portion 216 and 206 of proximal screw member 130, with portions 216 and 206 being slidably received within aperture 316 of portion 310. Aperture 316 has a diameter that is smaller than diameter 226 of bulbous portion 202.

Portion 310 of distal member 140 terminates into a second generally cylindrical portion 318 which has a plurality of threads 324, which are circumferentially disposed on the external surface of portion 318. Portion 318 has an internal circular aperture 326 which is longitudinally coextensive with portion 318 in direction 301. Circular aperture 326 aligns with aperture 312 to form a continuous opening from end 302 to end 304.

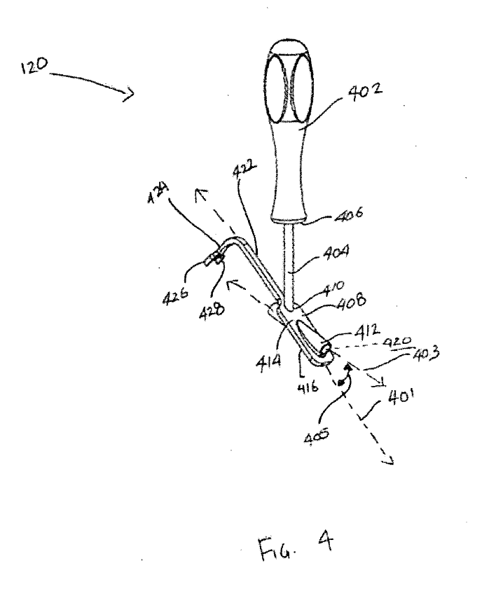

As shown in FIG. 4, instrument 120 is illustrated for coupling proximal screw member 130 to distal member 140. Particularly, instrument 120 includes a handle portion 402 coupled to a rod portion 404. Rod portion 404 emanates from handle portion 402 at end 406 and terminates into a rectangular planar portion 408 at end 410. Planar portion 408 is aligned along axis 401 and is fixably coupled to a generally cylindrical tubular portion 412 (i.e., an aiming device). Portion 412 traverses portion 408 from top surface 414 to bottom surface 416. Further, tubular portion 412 is aligned along dissimilar axis 403, forming an angle 405 with axis 401. Also, tubular portion 412 has a through aperture 420 that longitudinally traverses portion 412 along axis 403.

Planar portion 408 is coupled to planar portion 422, with portion 422 having a width slightly smaller than width of portion 408. Portion 422 terminates into a generally “U-shaped” portion 424 with portion 424 being orthogonal to portion 422. Further, portion 424 has a plurality of substantially similar sides 426 and 428 which are provided to be slidably coupled to grooves 326 and 328 of distal member 140.

In operation, sides 426 and 428 of instrument 120 are received in respective grooves 326 and 328 of distal member 140, of FIGS. 3A-3B, thereby slidably coupling distal member 140 to instrument 120. In this position, axis 303 of aperture 316 is aligned along substantially the same axis as axis 403 of instrument 120. Proximal screw member 130 is coupled to distal member 140 by slidably coupling portions 206 and 216 through aperture 420 of tubular portion 412. Tubular portion 412 guides proximal screw member 130 through internal aperture 420 and into aperture 316 on surface 322 and may also guide a Kirschner wire (K wire) or a drill. Proximal screw member 130, of FIG. 2, travels into bone as portions 216 and 206 travel further through aperture 316 at end 302 until bulbous portion 202 is restrained by surface 322 and end 302. Aperture 316, being tapered along axis 303, causes proximal screw member 130 to form an angle 150 with distal member 140, with proximal member 130 being aligned along an axis 303, which is substantially the same axis as axis 403 of tubular portion 412 of instrument 120.

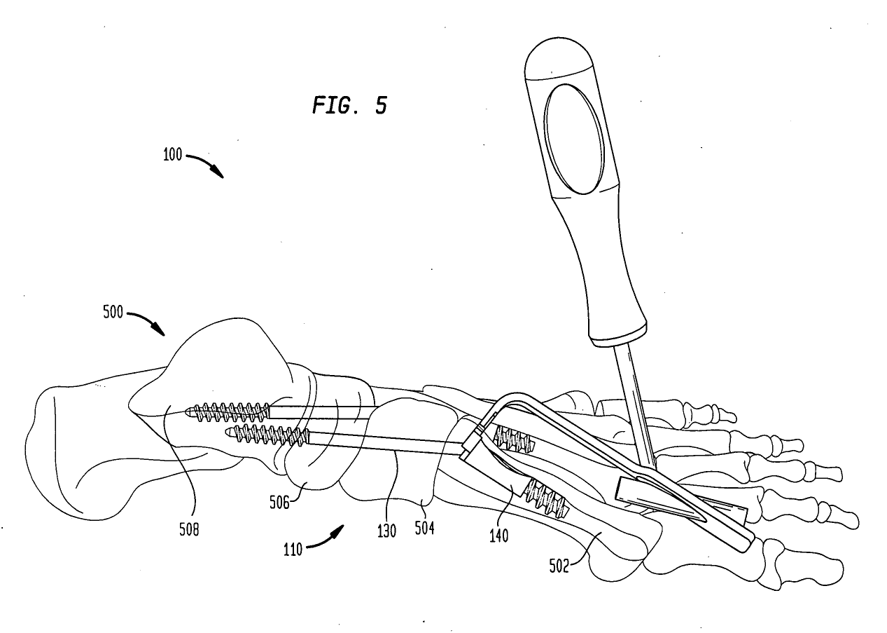

In operation, and as best shown in FIGS. 5, 6 and 7, the fixation system 100 utilizes the intramedullary fixation assembly 110 for treating and fixating the deteriorated and damaged or fractured bones in the human foot 500. This restores the arch in a human foot 500 by coupling the intramedullary fixation assembly 110 to the human foot 500 of a left leg. In one-non limiting example, and as shown in FIG. 5, the intramedullary assembly 110 is coupled to the medullary canals of the first metatarsal 502, medial cuneiform 504, navicular 506 and talus bone 508. Talus bone 508 makes up part of the ankle joint where the threaded portion 216 of the proximal screw member 130 of the intramedullary assembly 110 is threadably coupled. The medial cuneiform 504 and navicular 506 bones are most affected by Diabetic Charcot foot disorder that causes deterioration and collapse of the arch of the foot 500. It should be appreciated that the intramedullary assembly 110 may be used within each of the five rays, with a ray representing a line drawn from each metatarsal bone to the talus. The angulation in the smaller rays will be smaller than the two rays (i.e., a line from the first and second metatarsal bones to the talus bone). Also, the diameter of distal member 140 will decrease from the large ray to the small ray. In one non-limiting example, the angulation may be any angle greater than 90 degrees and less than 180 degrees. For example, the angle for the first ray may be 150-170 degrees and the angles for the other rays may be 160-175 degrees.

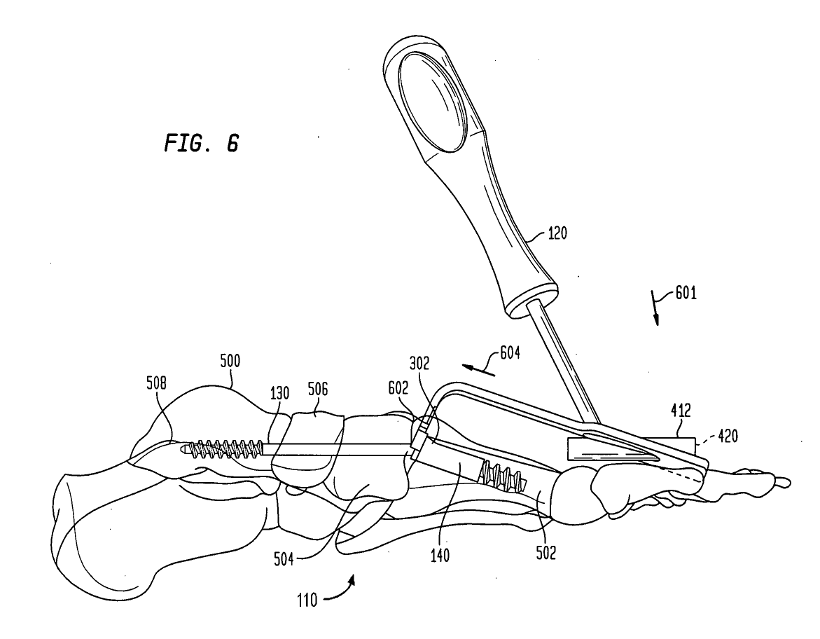

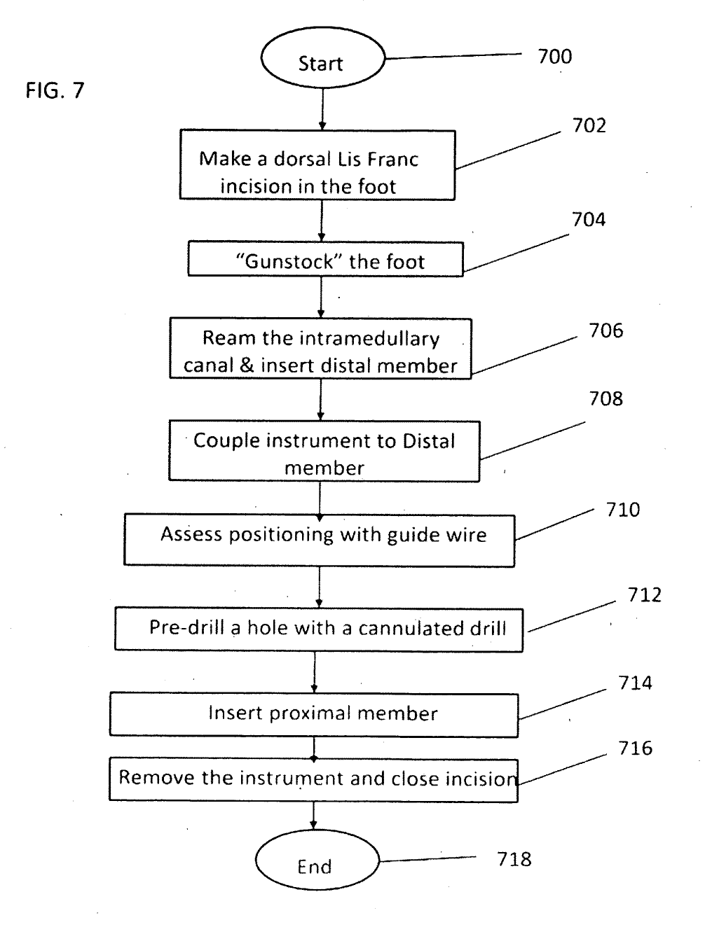

As shown in FIGS. 6 and 7, the intramedullary fixation assembly 110 may be utilized to reconstruct an arch in a mid-foot region of a human foot 500. As shown, the method starts in step 700 and proceeds to step 702, whereby a Dorsal Lis Franc incision (i.e., mid-foot incision) (not shown) is made in foot 500 in order to gain access to the joint. In step 704, the joint capsule is separated by “Gunstocking” foot 500 in direction 601 (i.e., the foot 500 is bent mid-foot) to expose the articular surface 602 and the articulating cartilage is removed. Next, in step 706, the intramedullary canal is reamed and the distal member 140 is inserted into the intramedullary canal (not shown) of the metatarsal 502. In other non-limiting embodiments, the distal member 140 may be inserted by impaction, by press fit, by reaming a hole in the intramedullary canal (not shown) or substantially any other similar strategy or technique.

Next, in step 708, the instrument 120 is coupled to the distal member 140 by coupling sides 426 and 428 of instrument 120 to respective grooves 326 and 328. In step 710, initial positioning of the proximal member 130 is assessed with the use of a guide wire through portion 412 (i.e., aiming device). Next, in step 712, a countersink drill is inserted through portion 412 and the proximal cortex is penetrated. In this step, a cannulated drill or guide wire is used to pre-drill the hole through the joints selected for fusion. In step 714, the proximal screw member 130 is inserted over the guide wire and into the distal member 140. Particularly, the proximal member 130 is inserted through tubular portion 412 (i.e., aiming device), causing proximal member 130 to travel through internal longitudinal aperture 420, into distal member 140 and further into bones 504, 506 and 508 until rigid connection with the tapered aperture 316 is made, thereby compressing the joint. In one non-limiting embodiment, a locking element (not shown) such as a plate or a washer is coupled to end 302 of the intramedullary fixation assembly 110 to further secure proximal threaded member 130 to distal member 140. Next, in step 716 the instrument 120 is removed and the dorsal Lis Franc (i.e., mid-foot) incision is closed. The method ends in step 718.

It should be appreciated that a plurality of intramedullary fixation assemblies, such as intramedullary fixation assembly 110, may be inserted into any of the bones of a foot 500 such as, but not limited to the metatarsal, cuneiform, calcaneus, cuboid, talus and navicular bones, in order to restore the natural anatomical shape of the arch of the foot 500. Thus, the fixation system 100, in one non-limiting embodiment, is utilized to couple the intramedullary fixation assembly 110 to the foot 500, which causes the metatarsal 504, medial cuneiform 504, navicular 506 and talus 508 bones to be aligned to the proper anatomical shape of an arch when assembled within foot 500. It should be appreciated that the intramedullary fixation assembly 110 is delivered through a dorsal midfoot incision, thereby reducing the disruption to the plantar tissues and/or the metatarsal heads while at the same time minimizing the tension on the skin. This allows for improved wound closure, reduced operating room time, reduction in the number of incisions required and reduction in the total length of incisions. It should also be appreciated that in other non-limiting embodiments, the intramedullary assembly 110 may be utilized with graft material (i.e., autograft, allograft or other biologic agent).

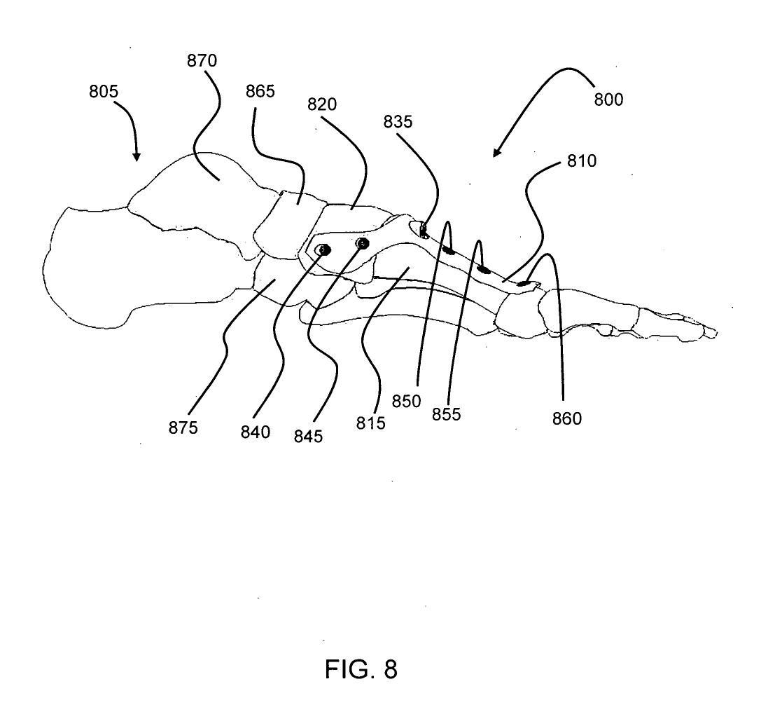

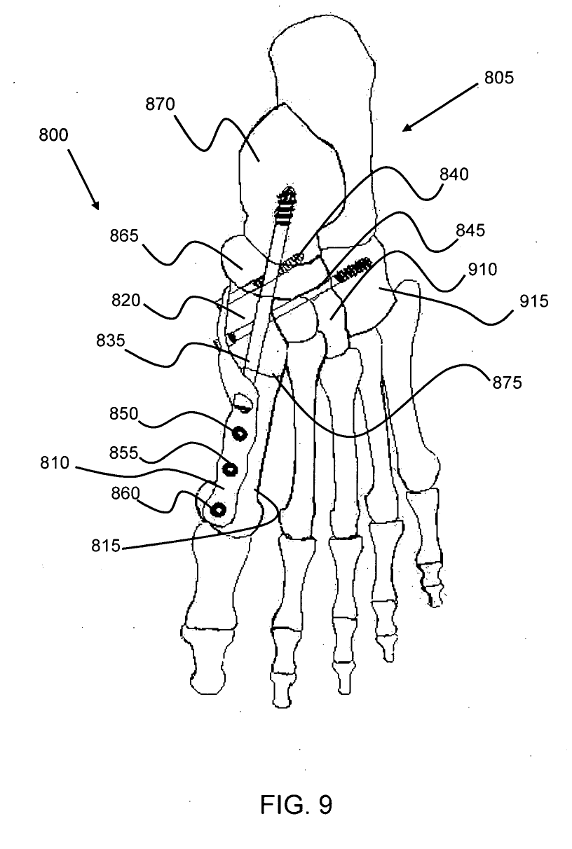

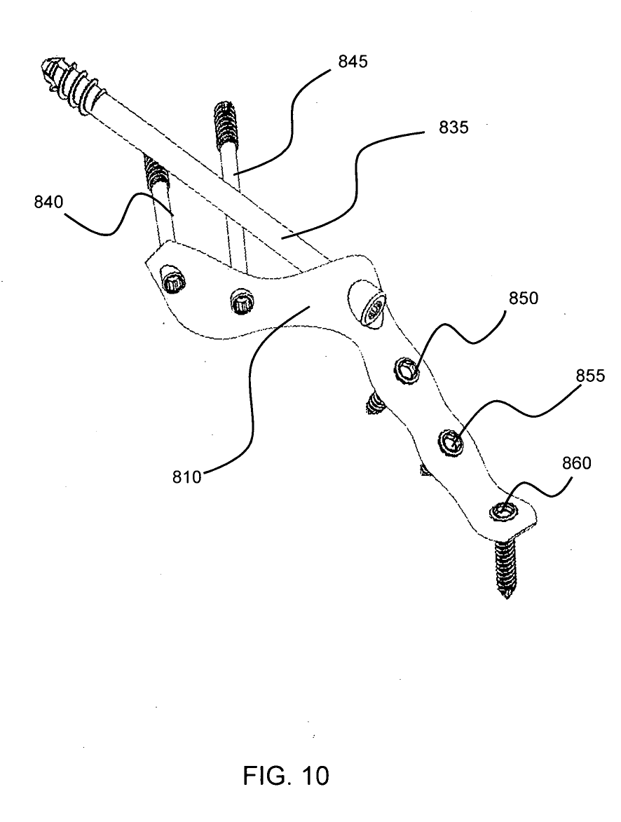

Referring now to FIGS. 8-10, there is shown a hybrid intramedullary fixation assembly 800 comprising a tarsometatarsal plate member 810 (hereinafter “TMT plate member 810”) and fixation screws, which is made in accordance with the teachings of an alternate embodiment of the invention. As shown, the TMT plate member 810 is anatomically designed for the first ray, however, the TMT plate member 810 may be designed to be accommodated on any of the other four rays in the human foot 805. The TMT plate member 810 is coupled to the human foot 805 (shown in FIGS. 8 and 9) through a combination of intramedullary screws 835, 840, 845 and polyaxial locking screws 850, 855, and 860, in order to reinforce the Tarsometatarsal Joint in the human foot 805. In other non-limiting embodiments, a non-locking screw may be utilized in lieu of the locking screws 850, 855, and 860. Further, the TMT plate member 810 receives the intramedullary screw 835 in order to couple the TMT plate member 810 to each of the metatarsal 815, the medial cuneiform 820, the navicular 865, and the talus 870 bones.

Further, and as shown in FIG. 9, the TMT plate member 810 is coupled to the medial cuneiform 820, intermediate cuneiform 905, the lateral cuneiform 910, and the cuboid 915 through a medial screw, such as, for example, the intramedullary screw 845. A second medial screw, such as, the intramedullary screw 840 is also coupled to the medial cuneiform 820, the navicular 875 (shown in FIG. 8), and the cuboid 915 to reinforce the tarsometarsal joint. The TMT plate member 810 also receives polyaxial locking screws 850, 855, and 860 in order to couple the TMT plate member 810 to the metatarsal 815. It should be appreciated that intramedullary screws 835, 840, 845 and polyaxial locking screws 850, 855, and 860 may vary in length in order to accommodate bones of varying sizes. It should also be appreciated that the intramedullary fixation assembly 800 may be used within each of the five rays, with a ray representing a line drawn from each metatarsal bone to the talus 870. The intramedullary fixation assembly 800, including the screws may be made from a Titanium material, although, in other non-limiting embodiments, intramedullary fixation assembly 800 may be made from SST, PEEK, NiTi, Cobalt Chrome or other similar types of materials. It should also be appreciated that intramedullary fixation assembly 800 may be utilized for the internal fixation of other bones in the human body, such as for example, the hand bones.

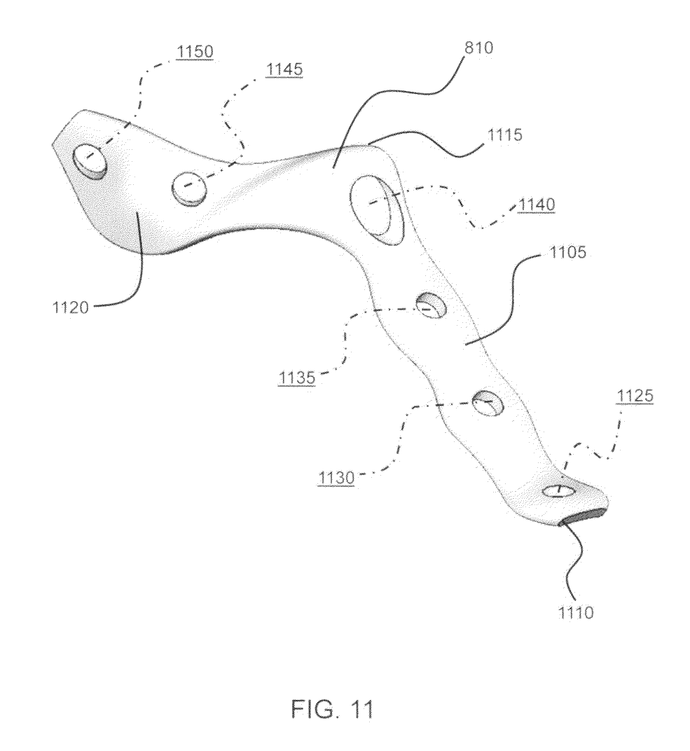

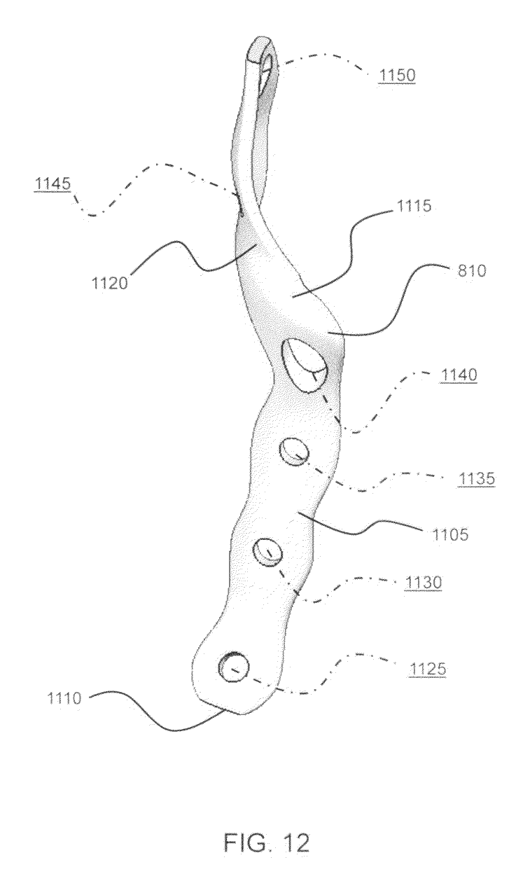

As shown in FIGS. 11-12, TMT plate member 810 has a generally “L-shaped” body. Particularly, TMT plate member 810 has a first generally flat and elongated member 1105 traversing from a first end 1110 to a second end 1115. End 1115 emanates and terminates into a second generally curved member 1120, which is provided to wrap around the medial cuneiform bone 820 (shown in FIGS. 8-9). End 1115 is further raised to accommodate the proximal head (not shown) of the metatarsal bone 815, although in other non-limiting embodiments, end 115 may be substantially flat to accommodate the other rays in the human foot 805 (shown in FIGS. 8-9). TMT plate member 810 further includes a plurality of holes 1125, 1130, and 1135 on the elongated member 1105, which are provided to receive a plurality of polyaxial locking screws 850, 855, and 860 (shown in FIGS. 8-10) in order to threadably couple TMT plate member 810 to the metatarsal 815 (shown in FIGS. 8-10). TMT plate member 810 further includes a plurality of holes 1140, 1145, and 1150, which are provided to receive intramedullary screw 835, 845, and 840 respectively (shown in FIGS. 8-9). In other non-limiting embodiments, a non-locking screw may be utilized in lieu of the locking screws 850, 855, and 860.

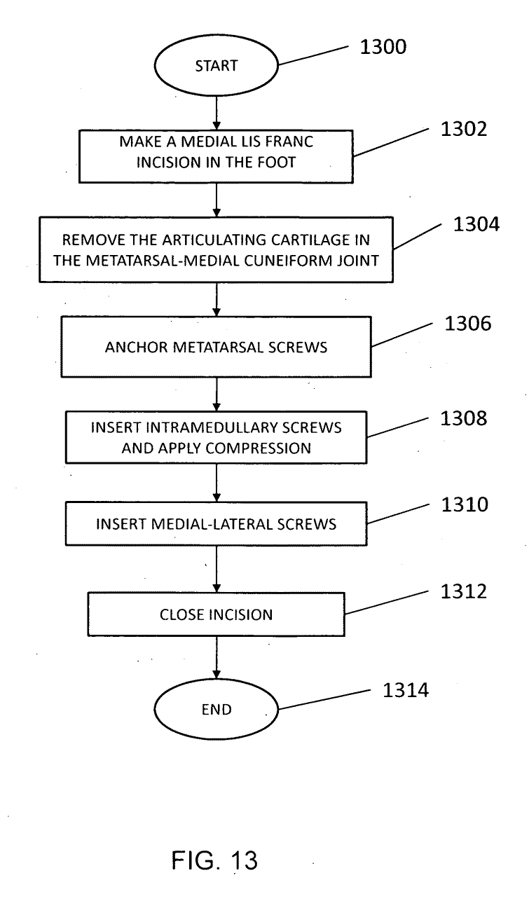

As shown in FIGS. 9 and 13, the intramedullary fixation assembly 800 may be utilized to reinforce the tarsal-metatarsal joint in a mid-foot region of a human foot 805. As shown, the method starts in step 1300 and proceeds to step 1302, whereby a Medial. Lis Franc incision (i.e., mid-foot incision) (not shown) is made in human foot 805 in order to gain access to the metatarsal 815 and medial cuneiform 820 bones. In step 1304, the joint capsule is separated by “Gunstocking” foot 805 (i.e., the foot 805 is bent mid-foot) to expose the articular surface 875 and the articulating cartilage is removed. Next, in step 1306, the TMT plate member 810 is positioned on top of the metatarsal 815 and the metatarsal screws, such as polyaxial locking screws 850, 855, and 860 are inserted into the metatarsal 815 and into the TMT plate member 810 to anchor the TMT plate member 810.

Next, in step 1308, initial positioning of the intramedullary screw 835 is assessed the intramedullary screw 835 is inserted into the human foot 805 and compression is applied to lock the TMT plate member 810 to the metatarsal 815, the medial cuneiform 820, the navicular 865, and the talus 870 bones. In one non-limiting embodiment, the positioning of the intramedullary screw member 835 is assessed with the use of a guide wire and a countersink drill is inserted to pre-drill a hole in the metatarsal 815 and medial cuneiform 820. Next, in step 1310, medial-lateral screws, such as screws 840 and 845 are inserted to reinforce the tarsometatarsal joint by locking the TMT plate member 810 to the medial cuneiform 820, intermediate cuneiform 905, the lateral cuneiform 910, the cuboid 915, and the navicular 875 respectively. Next, in step 1312, medial Lis Franc (i.e., mid-foot) incision is closed. The method ends in step 1314.

It should be appreciated that a plurality of intramedullary fixation assemblies, such as intramedullary fixation assembly 800, may be inserted into any of the bones of a human foot 805 such as, but not limited to the metatarsal, cuneiform, calcaneus, cuboid, talus and navicular bones, in order to stabilize the joints in the foot 805. It should be appreciated that the intramedullary fixation assembly 800 is delivered through a medial midfoot incision, thereby reducing the disruption to the plantar tissues and/or the metatarsal heads while at the same time minimizing the tension on the skin. This allows for improved wound closure, reduced operating room time, reduction in the number of incisions required and reduction in the total length of incisions. It should also be appreciated that in other non-limiting embodiments, the intramedullary assembly 800 may be utilized with graft material (i.e., autograft, allograft or other biologic agent).

It should be understood that this invention is not limited to the disclosed features and other similar method and system may be utilized without departing from the spirit and the scope of the invention.

While the invention has been described with reference to the preferred embodiment and alternative embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention, therefore, shall defined solely by the following claims. Further, it will be apparent to those of skill in the art that numerous changes may be made in such details without departing from the spirit and the principles of the invention. It should be appreciated that the invention is capable of being embodied in other forms without departing from its essential characteristics.

1. An intramedullary fixation assembly for joint stabilization, comprising:
a plate member having a plurality of apertures, wherein said plate member comprises a first elongated portion and a second curved portion; and
a plurality of metatarsal screws for coupling to said first elongated portion and for coupling to said metatarsal bone;
an intramedullary screw member coupled to said first elongated portion for applying compression; and
a plurality of medial screws coupled to said second curved portion for stabilizing said joint.

2. The intramedullary fixation assembly of claim 1, wherein said intramedullary screw member comprises a first elongated body, wherein said first elongated body includes a first threaded portion at a first end and a bulbous portion at a second end.

3. The intramedullary fixation assembly of claim 2, wherein said bulbous portion includes a taper for providing an interference fit with said plate member.

4. The intramedullary fixation assembly of claim 3, wherein said taper provides for an interference lock with said intramedullary screw member.

5. The intramedullary fixation assembly of claim 4, wherein said intramedullary screw member is cannulated having a circular cross-section with said first elongated body.

6. The intramedullary fixation assembly of claim 5, wherein said bulbous portion further includes an orifice longitudinally coextensive with a length of said bulbous portion.

7. The intramedullary fixation assembly of claim 6, wherein said orifice has a hexagonal shape, a star shape, or a square shape.

8. The intramedullary fixation assembly of claim 7, wherein said orifice is provided to receive a complementary shaped end of an instrument.

9. The intramedullary fixation assembly of claim 8, wherein said first threaded portion contains a plurality of bone threads on an outer surface of said threaded portion.

10. The intramedullary fixation assembly of claim 9, wherein said first threaded portion includes a self-tapping edge, wherein said self-tapping edge provides for removal of bone material during insertion of said intramedullary screw member.

11. The intramedullary fixation assembly of claim 10, wherein said first elongated portion is positioned on said metatarsal bone.

12. The intramedullary fixation assembly of claim 11, wherein said first elongated portion is threadably coupled to said metatarsal portion with said plurality of metatarsal screws for locking said first elongated portion to said metatarsal bone.

13. The intramedullary fixation assembly of claim 12, wherein said first elongated portion forms a predetermined angle with said second curved portion.

14. The intramedullary fixation assembly of claim 13, wherein said plurality of metatarsal screws are polyaxial locking screws.

15. The intramedullary fixation assembly of claim 13, wherein said plurality of metatarsal screws are non-locking screws.

16. A method for joint reinforcement, comprising:
providing an intramedullary fixation assembly, wherein the intramedullary fixation assembly further comprises:
a plate member having a plurality of apertures, wherein the plate member comprises a first elongated portion and a second curved portion;
a plurality of metatarsal screws coupled to the first elongated portion and coupled to the metatarsal bone;
an intramedullary screw member coupled to the first elongated portion for applying compression; and
a plurality of medial screws coupled to the second curved portion for stabilizing the joint.
making a medial lis franc incision;
removing an articulating cartilage from a metatarsal joint;
inserting the plurality of metatarsal screws into the metatarsal bone;
inserting the intramedullary screw member and applying compression to the metatarsal joint;
inserting the plurality of medial screws thereby stabilizing the metatarsal joint.

17. The method of claim 16, wherein the intramedullary screw member comprises a first elongated body, wherein the first elongated body includes a first threaded portion at a first end and a bulbous portion at a second end.

18. The method of claim 17, wherein the bulbous portion includes a taper for providing an interference fit with the plate member.

19. The method of claim 18, wherein the taper provides for an interference lock with the intramedullary screw member.

20. The method of claim 19, wherein the intramedullary screw member is cannulated having a circular cross-section with the first elongated body.

21. The method of claim 20, wherein the bulbous portion further includes an orifice longitudinally coextensive with a length of the bulbous portion.

22. The method of claim 21, wherein the orifice has a hexagonal shape, a star shape, or a square shape.

23. The method of claim 22, wherein the orifice is provided to receive a complementary shaped end of an instrument.

24. The method of claim 23, wherein the first threaded portion contains a plurality of bone threads on an outer surface of the threaded portion.

25. The method of claim 24, wherein the first threaded portion includes a self-tapping edge, wherein the self-tapping edge provides for removal of bone material during insertion of the intramedullary screw member.

26. The method of claim 25, wherein the first elongated portion is positioned on the metatarsal bone.

27. The method of claim 26, wherein the first elongated portion is threadably coupled to the metatarsal portion with the plurality of metatarsal screws for locking said first elongated portion to the metatarsal bone.

28. The method of claim 27, wherein said first elongated portion forms a predetermined angle with the second curved portion.

29. The method of claim 28, wherein the plurality of metatarsal screws are polyaxial locking screws.

30. The method of claim 28, wherein said plurality of metatarsal screws are non-locking screws.
